(19) United States
(12) Patent Application Publication
(10) Pub. No.: US 2009/0093429 A1
(43) Pub. Date: Apr. 9, 2009

(54) COMBINATION THERAPY FOR TREATING CANCER

(76) Inventors: Yang-Xin Fu, Chicago, IL (US); Ralph R. Weichselbaum, Chicago, IL (US)

Correspondence Address: MICHAEL BEST & FRIEDRICH LLP, ONE SOUTH PINCKNEY STREET, P O BOX 1806, MADISON, WI, 53701, US

(21) Appl. No.: 12/028,399

(22) Filed: Feb. 8, 2008

Related U.S. Application Data
(60) Provisional application No. 60/888,823, filed on Feb. 8, 2007.

(51) Int. Cl. A61K 31/7088; C12N 13/00; A61P 35/04

(52) U.S. Cl. 514/44; 435/173.1

(57) ABSTRACT

The invention relates to treatment of primary and secondary tumors using a therapeutic combination including ionizing radiation and gene therapy comprising mutant-LIGHT. Methods of treating a tumor and inducing an anti-tumor immune response are also provided.

### CROSS-REFERENCE TO RELATED APPLICATIONS

This application claims the benefit of U.S. provisional application 60/888,823 filed on Feb. 8, 2007. The provisional application is incorporated herein by reference.

### STATEMENT REGARDING FEDERALLY SPONSORED RESEARCH

This invention was made with government support under P01 CA097296 and R01 CA111423 awarded by the National Institute of Health. The government has certain rights in the invention.

### FIELD OF THE INVENTION

The invention relates generally to treatment of primary and secondary tumors using combined ionizing radiation and gene therapy. Adjunct therapies may also be used in combination with the invention.

### INTRODUCTION

Traditionally, cancer therapy has largely involved the use of surgery, chemotherapy and/or radiotherapy. Radiation therapy is an established cancer treatment employed in the treatment of approximately 60% of cancer cases. Radiation therapy is an important modality for treatment of human cancers, but it is often unsuccessful because of tumor cell radioresistance. Locoregional failure, the reappearance of cancer within the region it arose, also remains a significant problem after radiation therapy. This is partially due to the fact that metastasis in primary tumors can occur when the tumor is very small, and micrometastases can establish early in primary tumor development to seed distal tissue sites prior to clinical detection. Therefore, at the time of diagnosis, many cancer patients already have microscopic metastases. Additionally, local failure without metastasis accounts for significant cancer mortality, i.e. in brain, head and neck and cervical cancer.

Experiments in animal models, as well as clinical studies, indicate that the immune system can recognize and kill individual tumor cells. However, most naturally occurring T cell responses are not sufficient for either primary or metastatic tumor rejection by the host. The tumor microenvironment often forms barriers that prevent T cell priming, limit recruitment of T cells within the tumor and suppress incoming CD8+ T cells from becoming cytotoxic T lymphocytes (CTLs) against tumor antigens. Immunotherapy, in which the treatment triggers the body's immune system to recognize and respond to cancer cells, has been shown to elicit tumor-reactive T cells that can seek and destroy disseminated tumor antigen-positive cancer cells, but active vaccination for tumor bearing hosts has shown only limited benefit. Moreover, the lack of well defined antigens in most tumors limits the availability of vaccination or adoptive transfer therapy strategies.

Treating cancer with any single agent may have limited therapeutic efficacy. Several reasons account for the failure of single mode therapies. First, only a subset of the total population of tumor cells present is targeted, which leaves a subpopulation of cancerous cells to continue growing. Secondly, cells can develop resistance to a treatment after prolonged exposure. In contrast, combination therapies have been useful in circumventing resistance and increasing the target cell population.

LIGHT (TNFSF14) is a tumor-necrosis factor (TNF) family member that interacts with lymphotoxin β receptor (LTβR) and herpes virus entry mediator (HVEM) mainly expressed on stromal cells and T cells, respectively (Mauri et al. 1998, Immunity, 8, 21-30). LIGHT interaction with LTβR regulates chemokine expression. LIGHT also exhibits potent CD28-independent co-stimulatory activity for T cell priming and expansion, leading to enhanced T cell immunity against tumors and/or increased autoimmunity. Wild type LIGHT is not expressed on tumor cell surfaces, nor does it induce effective anti-tumor activity. Therefore, a form of LIGHT, designated mutant-LIGHT (LIGHT^{m}), has been generated to prevent protease digestion, resulting in LIGHT expression on tumor cells. Expression of LIGHT^{m} inside the tumor environment induces high levels of LTβR-associated chemokines and adhesion molecules that attract and prime naïve T cells leading to the rejection of established, highly progressive tumors in mice (US Patent Application Publication 2005/0025754, incorporated herein by reference).

### SUMMARY OF THE INVENTION

In one aspect, the invention provides a therapeutic combination for treating a tumor. The combination includes ionizing radiation and a construct comprising a sequence encoding LIGHT^{m}.

In another aspect, the invention provides a method of treating a subject bearing a solid tumor. The method includes administering ionizing radiation to a tumor cell and delivering a LIGHT^{m} construct to the tumor cell.

In still another aspect, the invention provides a method of enhancing anti-tumor T cell priming. The method includes administering ionizing radiation and delivering LIGHT^{m} construct to a tumor cell. Priming of anti-tumor T cells is enhanced using the combined treatment relative to either treatment alone.

In yet another aspect, the invention provides a method of enhancing radiosensitivity of a tumor. The method includes comprising delivering LIGHT^{m} construct to the tumor.

In an additional aspect, the invention provides a method of enhancing LIGHT^{m}-mediated tumor rejection comprising administering ionizing radiation to a tumor expressing LIGHT^{m}.

### BRIEF DESCRIPTION OF THE FIGURES

FIG. 1 is a histogram showing the expression of LIGHT^{m} on the surface of tumor cells after infection with Ad-LIGHT^{m} or Ad-control.

FIG. 2 is a graph showing the effects of intratumoral Ad-LIGHT^{m} treatment on primary tumors.

FIG. 3 is a bar graph showing the number of lung metastasis in mice injected with 4T1 mammary carcinoma cells and treated with surgery, Ad-LIGHT^{m}, Ad-control or Ad-LIGHT^{m} and anti-CD8 antibodies.

FIG. 4 is a graph showing survival rates of mice having resected primary tumors treated with Ad-LIGHT^{m} or Ad-control.

FIG. 5 is a line graph demonstrating the effects of radiation treatment on tumor growth and mouse survival.

FIG. 6 is a histogram demonstrating the distribution of anti-tumor specific T cells in draining lymph nodes and spleen of tumor-carrying mice after radiation treatment.

FIG. 7 are histograms demonstrating the distribution of activated anti-tumor specific T cells in draining lymph nodes (DN) and spleens (SP) of tumor-carrying mice. FIG. 7A and FIG. 7D are histograms demonstrating the distribution of activated anti-tumor specific T cells from mice treated with no radiation. FIGS. 7B and 7E are histograms demonstrating the distribution of activated anti-tumor specific T cells from mice treated with radiation alone. FIG. 7C and FIG. 7F are histograms demonstrating the distribution of activated anti-tumor specific T cells from mice treated with radiation and Ad-LIGHT^{m}.

FIG. 8A is a line graph showing the effects on tumor growth in mice of the combination treatment of Ad-LIGHT^{m} and radiation. FIG. 8B is a bar graph showing the number of metastatic tumors in tumor-bearing mice after combination treatment of radiation and Ad-LIGHT^{m}.

FIG. 9A is a line graph showing the effects of radiation and Ad-LIGHT^{m} treatment on tumor volume in mice with 4T1 tumors. FIG. 9B is a line graph showing the effects of radiation and Ad-LIGHT^{m} treatment on tumor volume in mice with B16-CCR7 tumors.

FIG. 10 is a line graph showing survival of mice with 4T1 tumors treated with combination of Ad-LIGHT^{m} and radiation therapy.

FIG. 11 is a bar graph showing the number of metastatic colonies in the lung of tumor-bearing mice treated with a combination of Ad-LIGHT^{m} and radiation therapy.

FIG. 12 is a bar graph showing the number of metastatic colonies in the draining lymph nodes of B16-CCR7 tumor bearing mice treated with a combination of Ad-LIGHT^{m} and radiation therapy.

FIG. 13A is a histogram depicting expression of MHC I on 4T1 cells irradiated with a low (5 Gy) dose of radiation. FIG. 13B is a histogram depicting expression of MHC class I on 4T1 cells after a high dose (15 Gy) of radiation. FIG. 13C is a histogram depicting expression of MHC class I on B16-CCR7 cells irradiated with a low (5 Gy) dose of radiation. FIG. 13D is a histogram depicting expression of MHC class I on B16-CCR7 cells after a high dose (15 Gy) of radiation. FIG. 13E is a histogram depicting expression of MHC class II on 4T1 cells irradiated with a low (5 Gy) dose of radiation. FIG. 13F is a histogram depicting expression of MHC class II on 4T1 cells after a high dose (15 Gy) of radiation. FIG. 13G is a histogram depicting expression of MHC class II on B16-CCR7 cells irradiated with a low (5 Gy) dose of radiation. FIG. 13H is a histogram depicting expression of MHC class II on B16-CCR7 cells after a high dose (15 Gy) of radiation.

FIG. 14 is a line graph depicting the proliferation of lymphocytes in vitro after co-culturing of Ad-LIGHT^{m} infected dendritic cells and uninfected lymphocytes.

### DESCRIPTION OF SEVERAL EMBODIMENTS

A variety of human and murine cancers have been proven to be antigenic and recognizable by T cells. However, peripheral solid tumors are not recognized by the immune system and these tumors are often termed “immunologically privileged.” This phenomenon may be due to the formation of immunological barriers within the tumor microenvironment that prevent T cell priming, limit recruitment of immune cells and suppress incoming CD8+ T cells from becoming CTLs. The present invention provides a means by which immune tolerance of both primary and secondary tumors can be overcome. The invention combines immunotherapy and radiotherapy and results in alteration of the tumor microenvironment to provide an effective treatment for a wide variety of tumors. In some embodiments, the invention effectively promotes formation of a lymphoid-like microstructure within the tumor where T cells can be recruited and activated after being presented with tumor specific antigens. The therapeutic combinations and methods provided herein, do not require prior determination of tumor-specific antigens.

Although the invention is not limited to action by any particular mechanism, delivery of LIGHT^{m} using a recombinant adenovirus (Ad-LIGHT^{m}) into the primary tumor may generate anti-tumor CTLs that exit the local tumor and patrol the periphery to eradicate spontaneous metastases. This strategy aims at utilizing the tumor tissue as a source of tumor antigens and also a site for recruitment and activation of CTLs in situ in a lymphoid-like microenvironment, providing a means to effectively mount an anti-tumor specific immune response against not only the primary tumor, but also metastases. Additional radiation treatment may induce apoptosis, anoikis or necrosis of primary tumor cells, which also provides a source of tumor antigens for T cell priming and expansion. This strategy provides a useful alternative to currently available therapies, as it relies on in vivo activation of CTLs against tumors.

In one embodiment, the invention provides a therapeutic combination for treating a tumor. The term “tumor” is used herein to describe an abnormal mass or growth of cells or tissue that is characterized by uncontrolled cell division. Tumors may be benign (not cancerous) or malignant (cancerous). The formation and growth of a tumor can be caused by acquired or inherited mutations to DNA within cells, which can result in a loss of control of cell division, cell differentiation, and/or cell growth. Tumors are identifiable through clinical screening or diagnostic procedures, including, but not limited to, palpation, biopsy, cell proliferation index, endoscopy, mammography, digital mammography, ultrasonography, computed tomography (CT), magnetic resonance imaging (MRI), positron emission tomography (PET), radiography, radionuclide evaluation, CT- or MRI-guided aspiration cytology, and imaging-guided needle biopsy, among others. Such diagnostic techniques are well known to those skilled in the art. Tumors that may be effectively treated by this invention include mammalian cancers, especially human cancers. Such treatment may be particularly useful in the treatment of, for example lung cancer, prostate cancer, ovarian cancer, testicular cancer, brain cancer, skin cancer, colon cancer, rectal cancer, gastric cancer, esophageal cancer, tracheal cancer, head and neck cancer, pancreatic cancer, liver cancer, breast cancer, ovarian cancer, lymphoid cancer, leukemia, cervical cancer, vulvar cancer, melanoma or sarcomas such as soft tissue sarcoma, osteosarcoma, chondrosarcoma and others. Tumor cell lines derived from the above cancers are also considered to be within the scope of the invention and may be used for purposes of study, for example, in mouse models of cancer treatment.

The combination of the invention includes ionizing radiation and a construct comprising a polynucleotide sequence encoding mutant-LIGHT (LIGHT^{m}). “Therapeutic combination” or “combination” in the sense of the present invention is to be understood as meaning that the individual components (i.e. the ionizing radiation and the construct) can be administered simultaneously or sequentially (directly one after the other or with a time interval of any suitable length) in the course of cancer treatment. The LIGHT^{m} construct can be administered before, during and/or after radiation treatment. Radiation treatment can be administered at any time so long as a beneficial effect from the co-action of the combination of the LIGHT^{m} construct and radiation treatment is achieved. Therapeutic combinations can also embrace further combination with adjunct therapies (such as, but not limited to, surgical tumor resection). The term “adjunct therapy” refers to any treatment administered in conjunction with the primary treatment and may include, but is not limited to, surgery, radiation therapy, hormone therapy, and/or biological therapy. However, it is to be understood that treatments that suppress the host's immune system would be detrimental to the combination treatment described herein because T cells are pivotal in eradicating tumor cells by way of the present invention.

The first component of the therapeutic combination is a construct encoding LIGHT^{m} (“LIGHT^{m} construct”). Native LIGHT is not expressed on tumor cells, nor does delivery of LIGHT sequences with viral vectors to tumor cells result in expression of LIGHT. Because native LIGHT has proteolytic sites in its sequence which may prevent its stable surface expression, a mutant version of LIGHT (designated LIGHT^{m}) was created. LIGHT^{m} is a form of the LIGHT protein that does not contain the proteolytic site EKLI at positions 79-82 in the native mouse amino acid sequence (SEQ. ID NO:1) and EQLI at position 81-84 in the native human amino acid sequence (SEQ ID NO:2) (see also, US Patent Application Publication 2005/0025754, incorporated herein by reference). SEQ ID NO:3 and SEQ ID NO:4 are the polypeptide sequences of LIGHT^{m} for mouse and human, respectively. After transduction of tumor cells with a LIGHT^{m} construct, expression may be detected on the surface of the tumor cells using an immunoglobulin fused to one of the LIGHT receptors, LTβR or HVEM.

The inventors have previously shown that LIGHT^{m} expression on tumor cells promotes tumor rejection. Introduction of LIGHT^{m} inside the tumor microenvironment elicits high levels of chemokines and adhesion molecules, accompanied by massive infiltration of naïve T lymphocytes which become primed against tumor antigens. LIGHT^{m} enhances rejection of an established, highly progressive parental tumor at local and distal sites. Tumor volume is reduced in vivo when LIGHT^{m} is expressed on tumor cells. Accordingly, therapeutic combinations of the invention include DNA constructs encoding LIGHT^{m}. A “construct” is an artificially constructed nucleic acid sequence that can be introduced into a target tissue or cells by way of, for example, a vector, including, but not limited to, plasmids, cosmids and viruses. Suitably, constructs include at least one polynucleotide encoding LIGHT^{m} operably connected to a regulatory sequence. A “regulatory sequence” is defined as a control sequence that modulates transcription of a nucleic acid, for example, a promoter. As used herein, “operably linked” or “operably connected” refers to a functional linkage between a regulatory sequence (such as a promoter or array of transcription factor binding sites) and a second nucleic acid sequence, wherein the regulatory sequence directs transcription of the nucleic acid corresponding to the secondary sequence. For instance, a promoter or enhancer is operably linked to a coding sequence if it affects the transcription of the coding sequence.

Delivery of constructs encoding LIGHT^{m} into a tumor may be direct, in which case the tumor is exposed to the construct in vivo, or indirect, in which case the tumor cells are obtained from e.g., a biopsy, transformed with the construct in vitro, and subsequently reintroduced into the patient. These approaches are routinely practiced in the art for suppressing tumors or treating other illness. Suitable in vitro and in vivo methods for administration of the construct containing LIGHT^{m} construct may include, but are not limited to, any method by which a polynucleotide (e.g., DNA) can be introduced into an organelle, a cell, a tissue or an organism as would be known to one of ordinary skill in the art. Suitable in vivo methods of administering the construct to the subject may include, but are not limited to, use of non-viral and viral vectors. Viral vectors suitable for delivering LIGHT^{m} DNA to tumor cells may include, but are not limited to, retroviruses (including lentiviruses), adenoviruses, adeno-associated viruses and herpes simplex virus type 1 or type 2. Most suitably, the construct is delivered to the tumor by intratumoral injection of the vector. In vitro delivery methods include, but are not limited to, transfection, including microinjection, electroporation, calcium phosphate precipitation, using DEAE-dextran followed by polyethylene glycol, direct sonic loading, liposome-mediated transfection and receptor-mediated transfection, microprojectile bombardment, agitation with silicon carbide fibers, desiccation/inhibition-mediated DNA uptake, transduction by viral vector, and/or any combination of such methods.

Suitably, a therapeutic dose of the construct is delivered to the tumor. A “therapeutic dose” or “therapeutically effective amount” refers to the amount of a construct or radiation administered that leads to enhanced survival or tumor regression within a subject. In some embodiments, a therapeutically effective dose of the construct used in combination with radiation is less than the amount that would be therapeutically effective if the construct were administered alone. It is common in cancer therapy to use the maximum-tolerated dose of each therapy, with a reduction only due to toxicity of the therapies used or potentiation of the toxicity of one therapy by the other. The construct may be administered by any suitable method that may be determined by one of ordinary skill in the art. For example, tumor cells can be transduced to express LIGHT^{m}. In vivo, the construct may be administered in an amount effective to prevent further proliferation of tumor cells and/or to cause regression of the tumor, without being overly toxic to the cell or subject. Expression of LIGHT^{m} within a tumor induces, for example, high levels of expression of chemokines and adhesion molecules and recruitment and priming of anti-tumor T cells within the tumor microenvironment. The construct may be delivered to the subject in a number of doses over a period of time. For example, the construct is delivered to the subject in about six doses over a 7 to 21 day period. In another suitable embodiment, the construct is delivered to the subject in about six doses over a 7 to 70 day period. In another suitable embodiment, the construct may be delivered in three to six doses once a week for 21 to 42 days. An example of a suitable dose is 1×10⁹ PFU of Ad-LIGHT^{m} delivered intratumorally.

The other component of the therapeutic combination is ionizing radiation. Radiation may be electromagnetic or particulate in nature. Electromagnetic radiation useful in the practice of this invention includes, but is not limited to, x-rays and gamma rays. Particulate radiation useful in the practice of this invention includes, but is not limited to, electron beams, proton beans, neutron beams, alpha particles, and negative pimesons. The unit of absorbed dose is the gray (Gy) which is defined as the absorption of 1 joule per kilogram. As is appreciated by those of skill in the art, the energy of the radiation determines the depth of absorption as well as the nature of the atomic interaction. Radiotherapy can be administered by a conventional radiological treatment apparatus and methods, or by intraoperative and sterotactic methods. Radiation may also be delivered by other methods that include, but are not limited to, targeted delivery, systemic delivery of targeted radioactive conjugates and intracavitary techniques (brachytherapy). Other radiation methods not described above can also be used to practice this invention.

Radiation therapy is suitably administered in a dose effective for the particular cancer to be treated, as determined by a person of ordinary skill in the art. The dose of radiation used in conjunction with the LIGHT^{m} construct may be similar to the amount administered when radiation is used alone, or, in some embodiments, may be reduced. In some cases, the dosage of radiation may be determined in relation to tumor volume and may depend on the type of tumor being treated. The dosage may also take into account other factors that can be determined by an ordinarily skilled clinician. Radiation treatment may be given as fractionated doses or as a bolus dose. For example, radiation is suitably administered 3 to 5 times per week, at least once a day, twice a day, or three times a day, with each treatment comprising 100 to 2000 centiGray (cGy) per dose. Treatment can be administered for 3-8 consecutive or non-consecutive weeks. Whether given as a bolus or as fractionated doses, total dose of radiation may be, for example, about 1000-10000 cGy. These are just examples of radiation treatment protocols, and this invention encompasses other treatment protocols that may be determined by a clinician of ordinary skill in the art.

Actual dosage levels of the construct and radiation may be varied so as to obtain the desired therapeutic response for a particular subject, composition and mode of administration, without being toxic to the subject. The selected dosage level will depend upon a variety of factors, including the route of administration, the rate of breakdown of the active form of the construct, the duration of treatment, other drugs, compounds, and/or materials used in combination with the particular construct, the age, sex, weight, condition, general health and prior medical history of the patient being treated, and like factors well known in the medical arts.

A benefit of lowering the dose of radiation as provided by the present invention includes a decrease in the incidence of adverse effects associated with higher dosages. By lowering incidence of adverse effects, an improvement in the quality of life of a patient undergoing treatment for cancer is contemplated. Further benefits of lowering the incidence of adverse effects include an improvement in patient compliance and a reduction in the number of hospitalizations needed for the treatment of adverse effects. Alternatively, the methods and combinations of the present invention can also maximize the therapeutic effect at higher doses.

The therapeutic combination of the invention can be combined with other cancer therapies, including, but not limited to, adjunct therapies (such as, but not limited to, surgical tumor resection and chemotherapy). For example, resection is generally a standard procedure for the treatment of tumors and cancers. The types of surgery that may be used in combination with the present invention include, but are not limited to, preventative, diagnostic or staging, curative and palliative surgery, and any other method that would be contemplated by those of skill in the art.

A further embodiment of the invention provides a method of enhancing survival of a subject bearing a solid tumor. The method includes administering ionizing radiation and delivering LIGHT^{m} construct to one or more cells within the tumor. The “subject” or “patient” to be treated may be any mammal including, but not limited to, humans, mice, rats, pigs, dogs, etc. The “solid tumor” treated in accordance with the method is a tumor that does not contain cysts or fluid. Such tumors include but are not limited to, sarcomas, carcinomas, and lymphomas. In some embodiments, the solid tumor is a primary tumor. A “primary tumor” is a tumor located in the first site it began to grow within the body. The primary tumor may be metastatic. A “metastatic tumor” is a tumor that can spread to other sites within the body to form metastases, micrometastates or secondary tumors. A “secondary tumor” is a tumor that has developed from the spread of cancer cells from the primary tumor to another site within the body. The primary and secondary tumors may be at different sites within the same organ, or may be located in different organs within the body.

In accordance with this embodiment of the invention, a tumor is treated in a subject. As used herein, tumor treatment encompasses any of the following: 1) inhibiting growth of the tumor, i.e., arresting its development, 2) preventing spread of the tumor, i.e., preventing metastases, 3) relieving the tumor, i.e., causing regression of the cancer, 4) preventing recurrence of the tumor, 5) palliating symptoms of the tumor, and 6) prolonging survival of the subject.

In some embodiments, the combination of LIGHT^{m} and radiation may be synergistic, i.e., their combined effect is greater than that predicted based on their individual activities. It is specifically envisioned that the combination of LIGHT^{m} and radiation act synergistically to inhibit tumor cell growth or increase tumor cell death and may be used to arrest or reduce proliferation of tumor cells, as described herein. Synergy between LIGHT^{m} construct and radiation may be evaluated by testing the agents separately and in combination for their ability to inhibit tumor cell growth and/or enhance survival of the patient.

Methods of treating a tumor may further include co-administration to the subject of an adjunct therapy, such as, but not limited to, surgery, as described above.

In still another embodiment, the invention provides a method of priming an anti-tumor T cell. The method includes administering ionizing radiation and delivering a LIGHT^{m} construct to a tumor cell. “Anti-tumor T cells” are T cells that have been activated against specific tumor antigens. Anti-tumor T cell “priming” encompasses the processes by which naïve T cells are activated against tumor antigens to proliferate, and further encompasses processes by which resting or memory anti-tumor T cells are reactivated to proliferate. “Enhanced proliferation,” as used herein, refers to an increase in the number of anti-tumor T cells in response to the combination therapy relative to the number of T cells generated using only Ad-LIGHT^{m} treatment or radiation therapy alone. The enhanced proliferation may be attributed to in situ expansion of naïve or activated T cells through costimulation and/or recruitment of additional T cells into the tumor tissue. Radiation may cause apoptosis of tumor cells, leading to an increase of tumor antigens in the tumor microenvironment that can prime and activate T cells. Enhanced production of cytokines and upregulation of adhesion molecules includes, but is not limited to, production of cytokines and adhesion molecules that attract and prime naïve T cells, e.g. CCL21, MAdCAM-1, and interferon-γ-induced chemokines. “Enhancing surface expression of receptors”, refers herein to an increase in expression of receptors and co-stimulatory molecules on the cell surface of T cells, lymphocytes, antigen presenting cells, or tumor cells. Expression of LIGHT^{m} on tumor cells upregulates the production of chemokines and expression of adhesion molecules in tumor tissue, resulting in the recruitment of naïve T cells that are then efficiently activated to tumor antigens and expanded within the tumor. The ability of LIGHT^{m} to provide CD28-independent costimulation results in selective and effective activation, expansion, and maintenance of tumor-specific T cells. Addition of radiation to LIGHT^{m} treatment may result in synergistic enhancement of activation and expansion of tumor-specific T cells.

Yet another embodiment of the invention is a method of enhancing radiosensitivity of a tumor by delivery of a LIGHT^{m} construct to the tumor. “Radiosensitivity” is defined herein as the relative susceptibility of cells, tissues, organs or organisms to the effect of ionizing radiation. Enhancement of radiosensitivity is herein defined as the increased ability of radiation treatment to result in tumor cell killing via apoptosis, necrosis, or anoikis.

Another embodiment of the invention is a method of enhancing LIGHT^{m}-mediated rejection of a tumor by administering ionizing radiation to a tumor expressing LIGHT^{m}. CD8+ T cells, or cytotoxic T lymphocytes that are primed against tumor antigens, are thought to play an intrinsic role in the ability of a host's immune system to attack tumor cells. LIGHT^{m} expression in tumors has been shown to increase the number of CD8+ T cells within the tumor microenvironment. The CD8+ T cells against tumor antigens may proliferate within the tumor microenvironment after LIGHT^{m} expression. An enhancement of tumor rejection by Ad-LIGHT^{m} in combination with radiation may be characterized as a reduction in tumor size, inhibition of tumor growth, reduction of tumor spread or metastasis, and/or increase in tumor cell killing as described above, relative to treatment with either alone. Radiation may act synergistically with Ad-LIGHT^{m} to enhance tumor rejection.

### EXAMPLES

### Example 1

Treatment with Ag104L^{d}-LIGHT^{m} cells eradicates established tumors at distal sites.

Either 1×10⁴ or 1×10⁶ (primary) Ag104L^{d} tumor cells were inoculated with or without 5×10⁴ (distal) Ag104L^{d} tumor cells into the left and right flank of C3B6F1 mice, respectively, and outgrowth was permitted for 14 or 20 days. Subsequently, 1×10⁶ Ag104L^{d} tumor cells transduced with retrovirus containing LIGHT^{m} or PBS were injected subcutaneously into the upper back (at a third site) with or without surgical resection of the primary tumor. As shown in Table 1, all mice treated with Ag104L^{d}-LIGHT^{m} tumor cells rejected the well-established Ag104^{d} parental tumors, while all control mice died as a result of uncontrolled distal tumor outgrowth.

TABLE 1

| Ag104L^{d} tumor | | | Days after | Incidence |
|---|---|---|---|---|
| cells injected | | | Ag104L^{d} | of tumor |
| Site 1 | Site 2 | Treatment | inoculation^{b} | growth (%) |
| 10⁴ | None | No treatment | 20 | 4/4 (100) |
| 10⁴ | None | 10⁶ Ag104L^{d}-LIGHT^{a} | 20 | 0/4 (0) |
| 10⁶ | 5 × 10⁴ | Surgical removal of tumor | 14 | 6/6^{c} (100) |
| | | at site 1 | | |
| 10⁶ | 5 × 10⁴ | Surgical removal of tumor | 14 | 0/7^{c} (0) |
| | | at site 1 & 10⁶ | | |
| | | Ag104L^{d}-LIGHT at a | | |
| | | third site | | |

### Example 2

Expression of LIGHT^{m} on tumor cell lines.

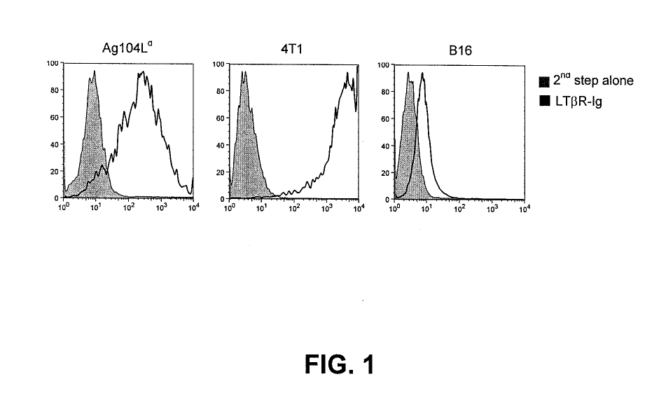

Mammary carcinoma 4T1, fibrosarcoma Ag104L^{d}, and melanoma B16 cells were plated at 3×10⁵ cells per well in a 6 well plate and infected in vitro with Ad-LIGHT^{m} or Ad-control (1×10⁸ PFU). Cells were harvested 24 hours after infection and stained for LIGHT^{m} expression with LTβR-Ig and human-IgG-PE. Flow cytometric analysis was performed 24 hours later. The data is a representative example of results. FIG. 1 demonstrates all three cell lines showed expression of LIGHT on the surface as seen by LTβR binding.

### Example 3

Inhibitory affects of Ad-LIGHT^{m} treatment on growth of primary tumors.

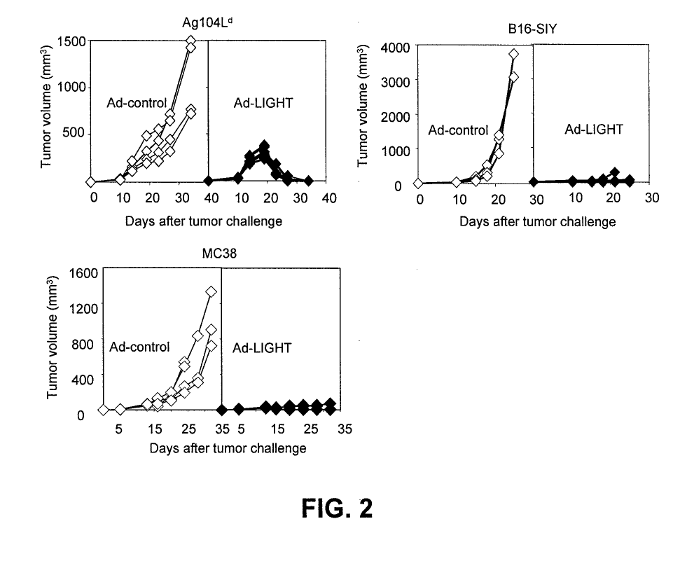

B6 wild type mice were injected subcutaneously with 1×10⁶ B16 (melanoma) cells, 1×10⁵ MC38 (colon adenocarcinoma) cells or Ad104L^{d} (fibrosarcoma) cells at a site 0.5-1 cm above the base of the tail. At either day 10 or day 13, 2×10⁹ PFU of either Ad-LIGHT^{m} or Ad-control were injected intratumorally. Tumor growth was measured every 3 to 4 days with a caliper. Size in cubic centimeters was calculated by the formula V=πabc/6, where a, b, and c are three orthogonal diameters. FIG. 2 shows mice bearing either the B16 tumor, MC38 tumor, or Ad104L^{d} tumor displayed tumor growth inhibition following treatment with Ad-LIGHT^{m}.

### Example 4

Ad-LIGHT^{m} treatment can reduce metastasis, and is CD8+ T cell dependent.

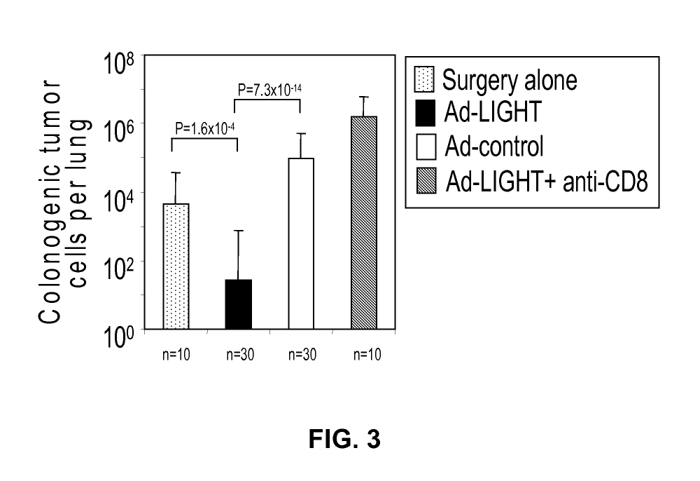

1×10⁵ 4T1 mammary carcinoma cells were injected subcutaneously into the flank of Balb/c mice, where it spontaneously metastasized at day 10. At day 14 and 17 post tumor inoculation, mice were treated intratumorally with 1×10⁹ PFU Ad-LIGHT^{m} or Ad-control. One group of mice was treated with surgery alone at day 14 post tumor inoculation. Another set of mice were CD8 depleted by injection of 125 μg/mouse anti-CD8 (YTS.169.4.2) interperitonally once per week starting at Day 14 after tumor inoculation. More than 90% of CD8+ T cells were depleted by this regime, as confirmed by flow cytometry staining of peripheral blood. Except for the mice treated with surgery alone, the primary tumors (˜150 mm3) on the mice were surgically resected on day 24 and mice were sacrificed for colonogenic lung assay on day 35. Colonogenic lung assay was performed as follows. The lung was removed from the mouse and transfered to a 6 well plate. 200 μl of collagenease medium (DMEM with 5% FBC and 1.5 mg/ml collagenase) was added to the lung and the lung was minced into small pieces and transferred to a 50 ml conical tube containing 5 ml of colleganase medium. The well was rinsed with 5 ml of collegenase medium to remove any small pieces of lung and added to the tube. The minced lung was incubated in a shaking incubator for 20 minutes at 37° C. at 175 rpm, and then lung solution poured through a cell strainer into a clean 50 ml conical tube. Any lung pieces left in the cell strainer underwent a second digestion. The collected digested cell suspension was spun at 1500 rpm for 5 minutes and the supernatant discarded. The pellet was resuspended in 1 ml collegenase-free medium (DMEM with 5% FBC) and underwent ACK lysis for 5 minutes. The cells were counted and 3×10⁵, 3×10⁴, and 3×10³ cells were plated into each well of a 12 well plate. 60 μM 6-thioguanine was added to each well, and the plate was incubated at 37° C., 5% CO₂ for 5-10 days. Culture media is removed and cells are fixed by adding 5 ml of methanol to each plate for 5 minutes (colonies should turn white). The methanol is removed and each well is rinsed gently with 5 ml distilled water. 5 ml of 0.03% (w/v) methylene blue solution is added to each plate and incubated for 5 minutes and then removed. The plate is rinsed gently with 5 ml distilled water and the plates are allowed to air dry before counting blue colonies, each colony representing one colonogenic metastatic cell. Data is a pool of multiple independent experiments. FIG. 3 shows Ad-LIGHT^{m} treatment significantly reduced the number of colonogenic tumor cells per lung compared with surgery alone or depletion of CD8+ T cells.

### Example 5

Ad-LIGHT^{m} treatment of mice enhances survival over surgery alone.

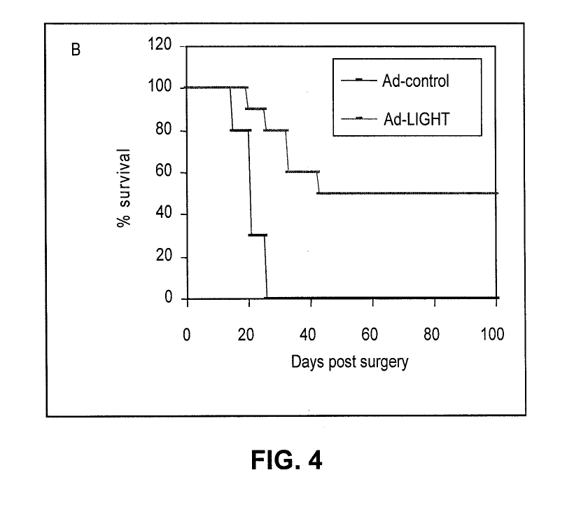

1×10⁵ 4T1 mammary carcinoma cells were injected subcutaneously into the flank of Balb/c mice. On day 14 or 17 post tumor inoculation, mice were treated intratumorally with 1×10⁹ PFU Ad-LIGHT^{m} or Ad-control. Primary tumors were surgically resected on Day 24. The mice were checked for survival at 100 days post surgery. FIG. 4 demonstrates that 50% of mice treated with Ad-LIGHT^{m} in combination with surgery survived long term.

### Example 6

Radiation can slow tumor growth but has little impact on survival.

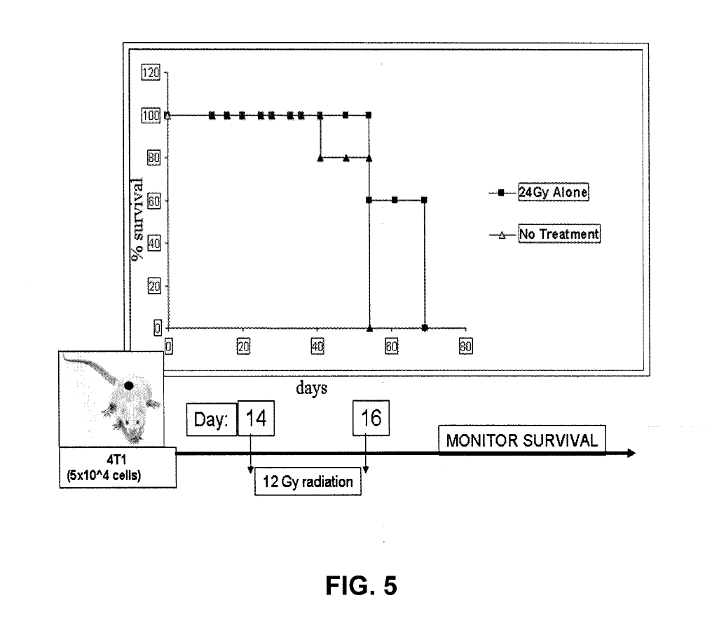

5×10⁴ 4T1 tumor cells were inoculated into Balb/c mice (n=10). On Day 14 and Day 16, the mice were irradiated with 12 Gy. Survival was monitored twice a week. FIG. 5 shows radiation did not increase survival alone, but it somewhat slowed the growth of the tumor.

### Example 7

Radiation and Ad-LIGHT^{m} enhance T cell proliferation in draining lymph nodes.

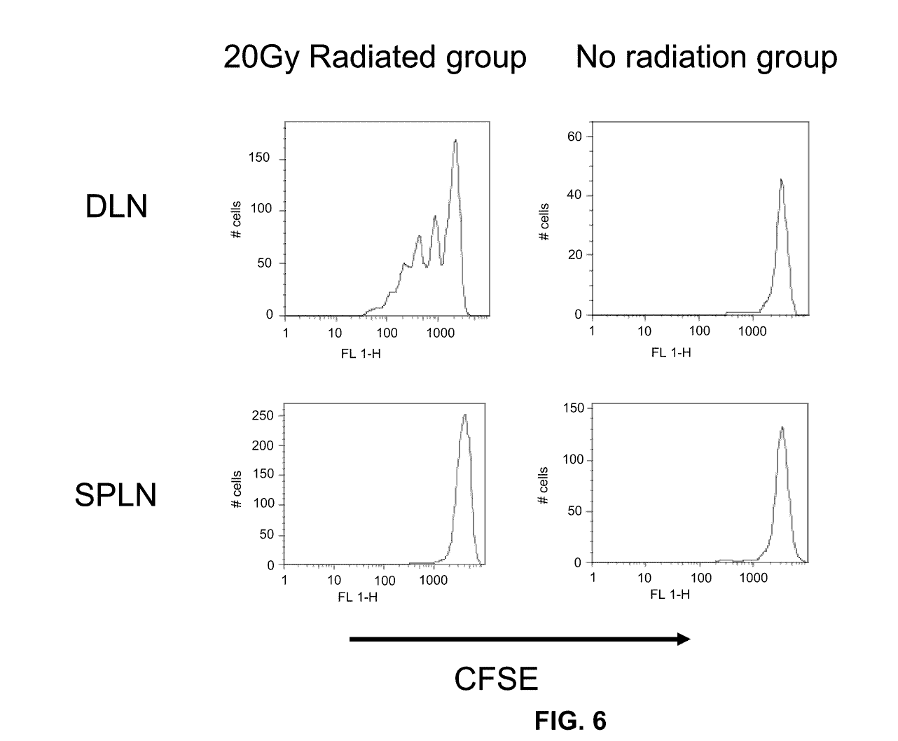

To test the ability of radiation to enhance T cell proliferation, B6 mice were inoculated with 1 to 5×10⁵ B16 tumor cells. 2×10⁶ Carboxyl-Fluoroscein diacetate, Succinimidyl Ester (CFSE) labeled Pmel T cells were adoptively transferred to B16 melanoma bearing mice. On day 14, the mice were divided into two groups, one group received 20 Gy local radiation and the other received no radiation. At Day 4, the spleen (SPLN) and draining lymph nodes (DLN) were removed from the mice and prepared for flow cytometry. FIG. 6 demonstrates that mice treated with radiation had increased anti-tumor T cells in draining lymph nodes.

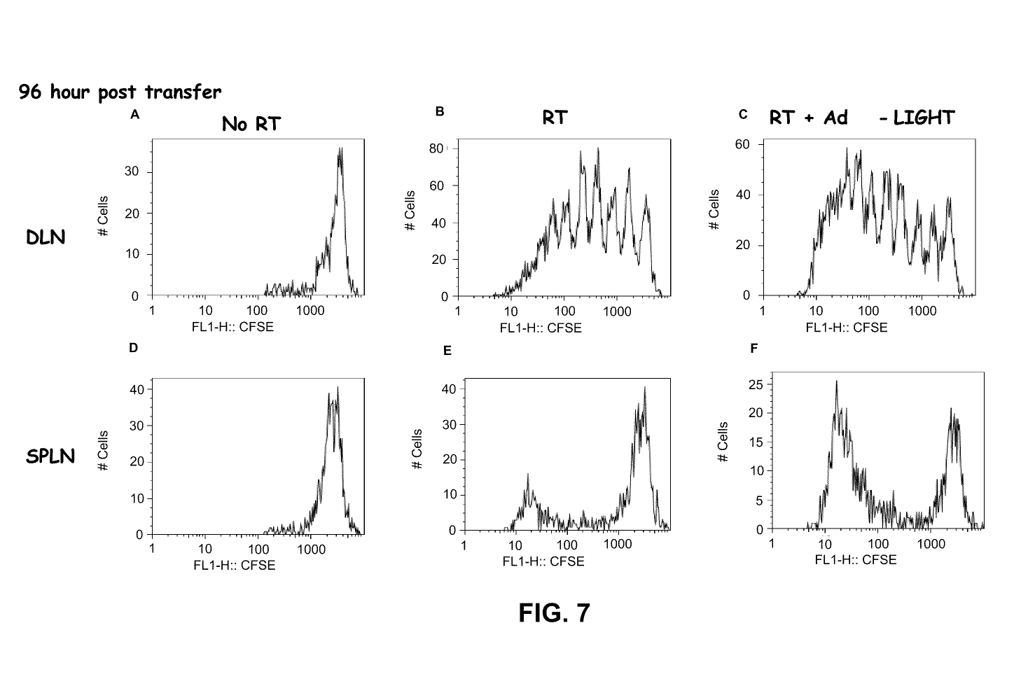

To test the ability of radiation and Ad-LIGHT^{m} to enhance T cell proliferation, B16-SIY tumors (5×10⁵ cells) were injected into the lower back of C57BL/6 mice. 14 days after tumor challenge, the mice were radiated with 20 Gy and intratumorally challenged with 4×10¹⁰ viral particles of Ad-control (ad-LacZ) or Ad-LIGHT^{m} adenovirus. CFSE labeled naïve 2C cells were adoptively transferred intravenously to the treated mice. 96 hours post-adoptive transfer, mice were sacrificed and their spleen (SPLN) and draining lymph nodes (DLN) were removed and prepared for flow cytometry. The transgenic T cells within the spleen and draining lymph nodes were analyzed by flow cytometry and the degree of CFSE dilution was determined by gating 1B2⁺CD8⁺ lymphocyte populations. FIG. 7 demonstrates the results of this experiment. As seen, radiation alone (FIGS. 7B and 7E) was able to induce proliferation of transgenic T-cells when compared with the untreated mice (FIGS. 7A and 7D). Radiation in combination with Ad-LIGHT^{m} (FIGS. 7C and 7F) enhanced proliferation of transgenic T-cells above that observed with radiation alone (FIGS. 7B and 7E) in both the spleen and draining lymph nodes.

### Example 8

Local Radiation with Ad-LIGHT^{m} controls tumor growth and rejects established tumors.

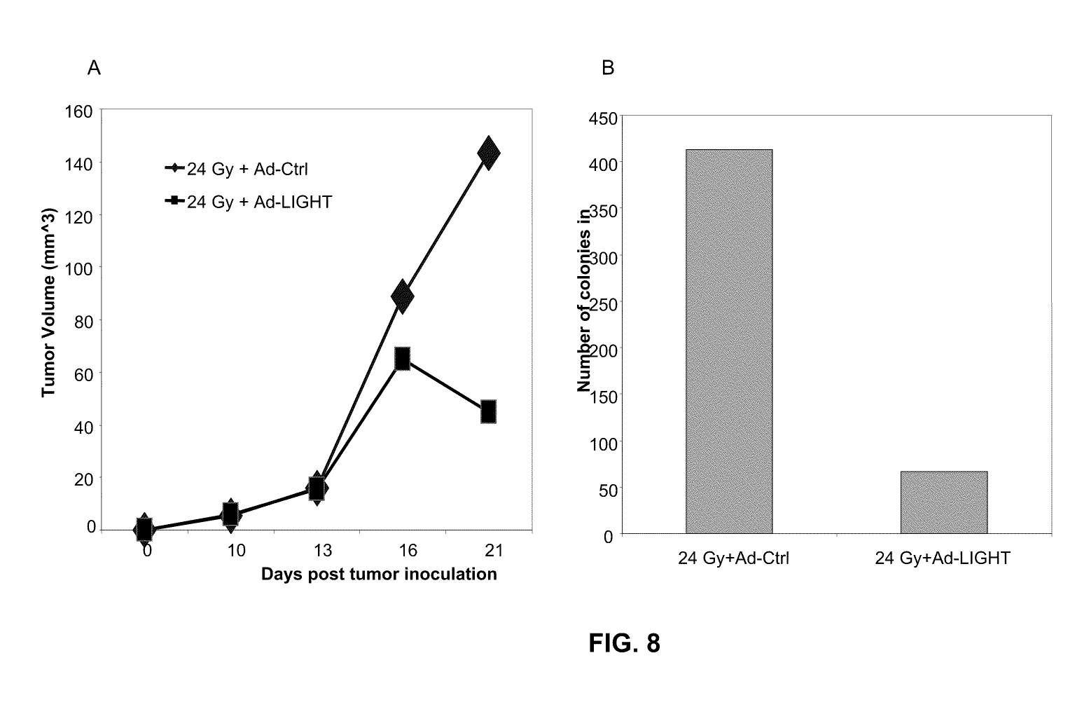

Eight mice were injected subcutaneously with 4×10⁵ B16-CCR7 tumor cells. All mice were treated with 12 Gy radiation on Day 9 and 10. The mice were split into two equal groups, and either received Ad-LIGHT^{m} or Ad-control on Day 10, Day 12 and Day 13. Tumors were removed on Day 16, and colony assays were performed on Day 23 as described in Example 4. FIG. 8 shows radiation treatment in combination with Ad-LIGHT^{m} leads to inhibition and regression of tumor growth and the reduction of metastasis in draining lymph nodes.

To test the combined efficacy of radiation and Ad-LIGHT^{m} on primary tumor growth of melanoma and breast cancer cells, C57BL/6 mice were injected in the lower back with 1×10⁵ B16-CCR7 melanoma cells or injected in the with 5×10⁴ 4T1 breast carcinoma cells. Mice from both the melanoma and breast cancer cells were divided into three treatment groups, radiation alone, adenovirus alone or combination of radiation and Ad-LIGHT^{m} treatment. The combination treatment groups were then locally radiated with 12 Gy on days 14 and 15, (24 Gy total) followed by intratumoral inoculation with 2×10¹⁰ virus particles of Ad-LIGHT^{m} or Ad-control on days 15 (concomitant with second dose of radiation) and 16. The melanoma treated mice had an additional inoculation on day 17. The radiation only group was radiated with 12 Gy of radiation on days 14 and 15. The adenovirus (Ad) only groups (Ad-LacZ or Ad-LIGHT^{m}) were inoculated intratumorally on days 15, 16 and 17. Control mice received radiation followed by Ad-control (Ad-LacZ) or no radiation and just adenovirus. Tumor measurements were taken twice weekly. Tumors were surgically resected in day 25 post tumor inoculation. Mice were monitored for survival.

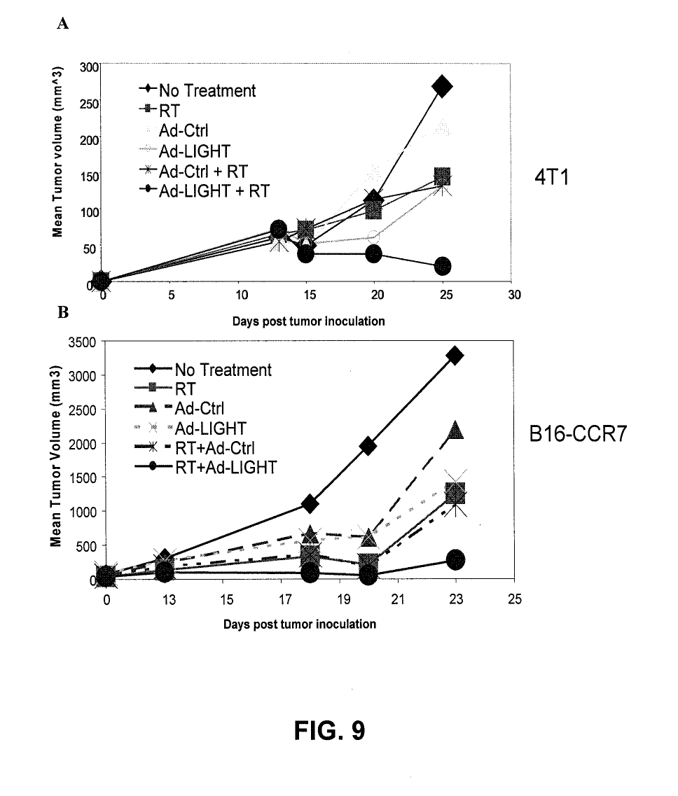

Untreated mice or mice treated with Ad-control (LacZ) demonstrated rapid tumor growth over the 25 day observation period. Mice receiving radiation alone, Ad-LIGHT^{m} alone, or no radiation followed by Ad-LIGHT^{m} or Ad-control demonstrated an approximately 50% reduction in tumor volume as compared to control mice for both melanoma and breast cancer cell treated mice (FIGS. 9A and 9B). Further, mice receiving both radiation and Ad-LIGHT^{m} were able to further control the growth of the primary tumor, with most mice surviving more than 100 days while control mice treated with radiation alone died at 60-70 days compared with untreated mice that died around 40-50 days.

### Example 9

Radiation and Ad-LIGHT^{m} treatment eradicates metastases leading to long term survival.

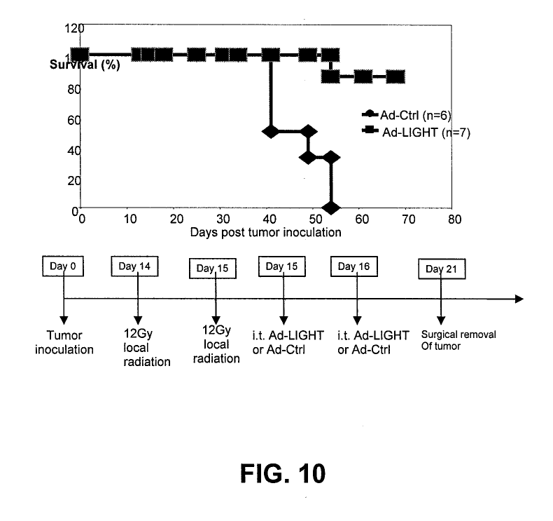

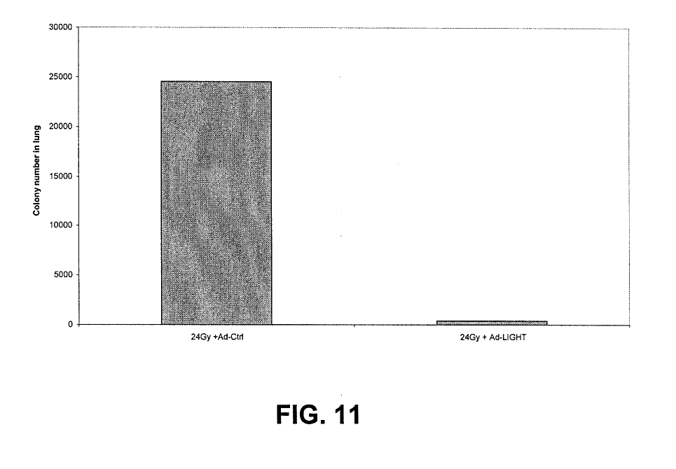

To test whether Ad-LIGHT^{m} in addition to radiation treatment can eradicate metastasis and improve survival, Balb/c mice were challenged with higly aggressive, spontaneously metastasizing 4T1 breast carcinoma. 5×10⁴ 4T 1 tumor cells were injected subcutaneously into the flank of Balb/c mice. Mice were irradiated on Day 14 and Day 15 post tumor inoculation with 12 Gy. 2×10¹⁰ Plaque Forming Units (PFUs) of Ad-LIGHT^{m} or Ad-control were injected intratumorally on Day 15 and 16 post-tumor inoculation. Primary tumors of the mice were surgically resected on Day 25. The mice were monitored twice a week over 100 days post-surgery. Some mice were sacrificed on Day 35 for colonogenic lung assay to assay for metastasis. FIG. 10 demonstrates that 85% of mice treated with Ad-LIGHT^{m} in combination with radiation (and surgery) survived long term while most mice treated with radiation (and surgery) alone died by Day 50. Without radiation, 50% mice died in 50 days as seen in FIG. 4. FIG. 11 shows that only 1 out of 3 combined treated mice had colonies in the lung and the number of colonies in the lung was lower in the combined treatment than radiation alone.

### Example 10

Radiation and Ad-LIGHT^{m} treatment eradicates melanoma metastases leading to long term survival.

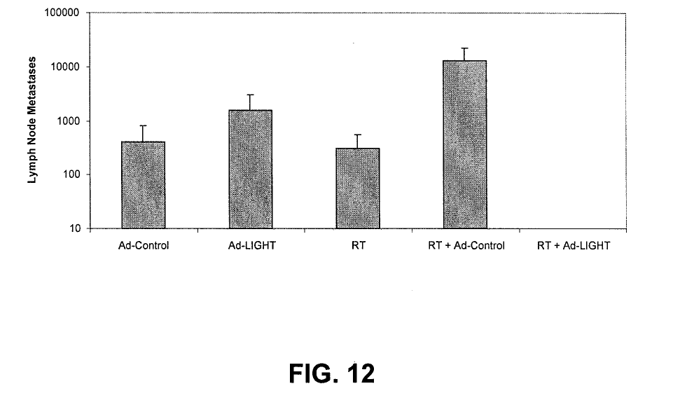

Melanoma cell line B16-CCR7 was received from Sam Huang at the National Cancer Institute (Bethesda, Md.). B16-CCR7 has similar metastatic kinetics as 4T1, spontaneously metastasizing to the draining lymph nodes by Day 11 post-inoculation. These cells were used to test the ability of radiation and Ad-LIGHT^{m} treatment to eradicate metastasis. B6 mice were inoculated subcutaneously with 1×10⁵ B16-CCR7 melanoma cells and given local radiation (12Gy) on Day 14 and Day 15 post-innoculation. 2×10¹⁰ virus particles of Ad-LIGHT^{m} or Ad-control were administered intratumorally on day 15, 16, and 17. Tumors were excised on day 25. Tumor colony assay on the draining lymph nodes (DLNs) was performed on day 35 similar to the method described in Example 4 except with draining lymph node tissue instead of lung tissue. As shown in FIG. 12, metastasis and progressive growth of B16-CCR7 tumors were resistant to both radiation and intratumoral Ad-LIGHT^{m} treatment alone. Mice treated with the combination of radiation and Ad-LIGHT^{m} had an absence of tumor colonies in the draining lymph nodes (FIG. 12). FIG. 12 presents data from two experiments.

### Example 11

### Radiation Treatment Increases Expression of MHC Class I and Costimulatory Molecules

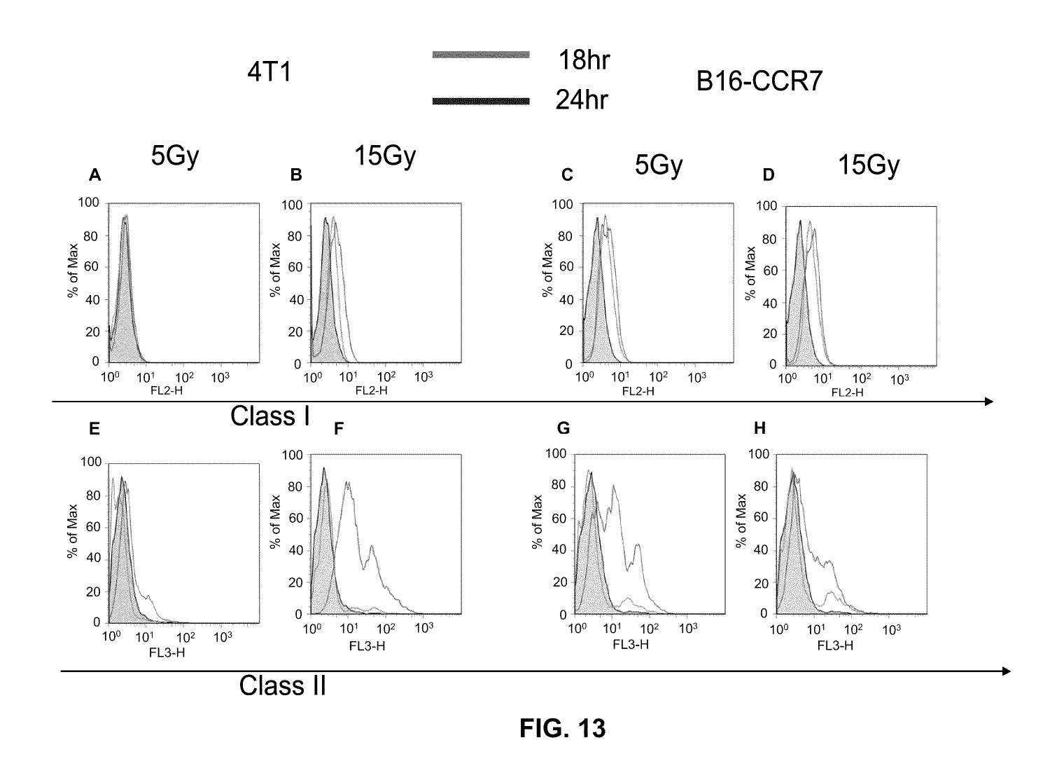

To determine whether radiation treatment could result in changes to the local tumor environment and enhance the effector phase of CTLs, the expression of key molecules involved in CTL effector function such as MHC class I and costimulatory molecules CD80 and CD86 were evaluated. For in vitro testing, 4T1 breast carcinoma and B16-CCR7 melanoma cells were radiated with either 5 Gy or 15 Gy radiation and then plated at a 1×10⁶ cells/well in a 6 well pate. Cells were harvested at 18 hours and 24 hours post-radiation, and stained for MHC class I/II, CD80 or CD86. FIG. 13 are the histograms depicting the expression of MHC class I and II on T41 tumor cells or B16-CCR7 tumor cells after low (5 Gy) or high (15 Gy) radiation treatment. FIGS. 13A and 13B depict MHC class I expression on T41 cells after low and high radiation treatment, respectively. FIGS. 13C and 13D depict MHC class I expression on B16-CCR7 cells after low and high radiation treatment, respectively. FIGS. 13E and 13F depict MHC class II expression on T41 cells after low and high radiation treatment, respectively. FIGS. 13G and 13H depict MHC class II expression on B16-CCR7 cells after low and high radiation treatment, respectively.

As the results show, irradiation of tumor cells in vitro either at low or high doses of radiation induced expression of MHC I and II, with the higher the dosage of radiation producing a more robust response. Similar results were seen with both CD80 and CD86, both key co-stimulatory molecules for CTL responses.

### Example 12

### Ad-LIGHT^{m} Expression on Dendritic Cells Increases T Cell Priming

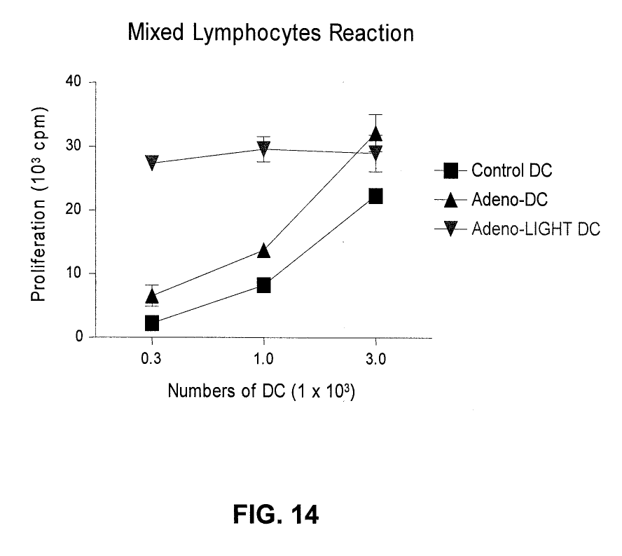

To test whether dendritic cells (DCs) expressing Ad-LIGHT^{m} stimulate T cell proliferation above that of control dendritic cells, bone marrow derived dendritic cells (BMDC) from B6 mice were infected in vitro with 1×10⁹ virus particles/ml of Ad-LIGHT^{m} or Ad-control overnight, and then co-cultured in a mixed lymphocyte reaction with Balb/c splenocytes as responders. DCs and splenocytes were cocultured form 5 days and ³H-thymidine was added for the last 18 hours. T cell priming was measured by a read out of ³H-thymidine incorporation. As shown in FIG. 14, DCs expressing Ad-LIGHT^{m} were able to stimulate T cell priming better than control DCs.

While the compositions and methods of this invention have been described in terms of exemplary embodiments, it will be apparent to those skilled in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention. In addition, all patents and publications listed or described herein are incorporated in their entirety by reference.

As used in this specification and the appended claims, the singular forms “a,” “an,” and “the” include plural referents unless the content clearly dictates otherwise. Thus, for example, reference to a composition containing “a polynucleotide” includes a mixture of two or more polynucleotides. It should also be noted that the term “or” is generally employed in its sense including “and/or” unless the content clearly dictates otherwise. All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

It also is specifically understood that any numerical value recited herein includes all values from the lower value to the upper value, i.e., all possible combinations of numerical values between the lowest value and the highest value enumerated are to be considered to be expressly stated in this application.

We claim:

1) A therapeutic combination for treating a tumor comprising:
a) ionizing radiation; and
b) a construct comprising a polynucleotide sequence encoding mutant-LIGHT operably connected to a promoter functional in a cell of the tumor.

2) The therapeutic combination of claim 1, wherein the construct further comprises the polynucleotide sequence in an adenoviral vector.

3) The therapeutic combination of claim 1 wherein the polynucleotide sequence encodes a polypeptide sequence comprising SEQ ID NO: 3.

4) A method of treating a solid tumor in a subject comprising:
a) administering ionizing radiation to the tumor; and
b) delivering a construct comprising a polynucleotide sequence encoding mutant-LIGHT operably connected to a promoter functional in a cell of the tumor.

5) A method of claim 4, wherein the solid tumor is a primary tumor.

6) The method of claim 5 further comprising resecting the primary tumor.

7) The method of claim 4, wherein the solid tumor is a secondary tumor.

8) The method of claim 7 further comprising resecting the secondary tumor.

9) The method of claim 4, wherein the solid tumor is a metastatic tumor.

10) The method of claim 4, wherein the tumor is selected from the group consisting of a breast tumor, a skin tumor, a lung tumor, a colon tumor, a prostate tumor, an ovarian tumor, a testicular tumor, a brain tumor, an esophageal tumor, a head and neck tumor, a pancreatic tumor, a liver tumor, a lymphoid tumor, a melanoma and a sarcoma such as a soft tissue sarcoma, osteosarcoma, chondrosarcoma or other sarcoma.

11) The method of claim 4 further comprising administering an adjunct therapy.

12) The method of claim 11, wherein the adjunct therapy is surgery.

13) The method of claim 4, wherein the mutant-LIGHT construct is delivered in a therapeutic dose.

14) A method of priming an anti-tumor T cell comprising:
a) administering ionizing radiation to a tumor cell; and
b) delivering a construct comprising a polynucleotide sequence encoding mutant-LIGHT operably connected to a promoter functional in a cell of the tumor.

15) The method of claim 14, wherein the T cell is a cytotoxic T lymphocyte (CTL).

16) The method of claim 14, wherein the construct further comprises the polynucleotide sequence in an adenoviral vector.

17) The method of claim 14, wherein the anti-tumor cell is in a subject.

18) A method of enhancing the radiosensitivity of a tumor cell comprising delivering a mutant-LIGHT construct to the tumor cell.

19) The method of claim 18, wherein the tumor cell is within a subject.

20) The method of claim 18, wherein the tumor is a selected from the group consisting of a breast tumor, a skin tumor, a lung tumor, a colon tumor, a prostate tumor, an ovarian tumor, a testicular tumor, a brain tumor, an esophageal tumor, a head and neck tumor, a pancreatic tumor, a liver tumor, a lymphoid tumor, a melanoma and a sarcoma such as a soft tissue sarcoma, osteosarcoma, chondrosarcoma or other sarcoma.

21) The method of claim 18, wherein the construct further comprises the polynucleotide sequence in an adenoviral vector.

22) The method of claim 18, wherein delivering the mutant-LIGHT construct is by intratumoral injection.

23) The method of claim 18, wherein the mutant-LIGHT construct is delivered in a therapeutic dose.


SEQUENCE LISTING (4 sequences)

SEQ ID NO: 1 - protein, 239 aa, Mus musculus
MESVVQPSVFVVDGQTDIPFRRLEQNHRRRRCGTVQVSLALVLLLGAGLATQGWFLLRLHQRLGDIVAHLPDGGKGSWEKLIQDQRSHQANPAAHLTGANASLIGIGGPLLWETRLGLAFLRGLTYHDGALVTMEPGYYYVYSKVQLSGVGCPQGLANGLPITHGLYKRTSRYPKELELLVSRRSPCGRANSSRVWWDSSFLGGVVHLEAGEEVVVRVPGNRLVRPRDGTRSYFGAFMV

SEQ ID NO: 2 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 3 - protein, 235 aa, Mus musculus
  misc_feature: mouse mutant-LIGHT
MESVVQPSVFVVDGQTDIPFRRLEQNHRRRRCGTVQVSLALVLLLGAGLATQGWFLLRLHQRLGDIVAHLPDGGKGSWQDQRSHQANPAAHLTGANASLIGIGGPLLWETRLGLAFLRGLTYHDGALVTMEPGYYYVYSKVQLSGVGCPQGLANGLPITHGLYKRTSRYPKELELLVSRRSPCGRANSSRVWWDSSFLGGVVHLEAGEEVVVRVPGNRLVRPRDGTRSYFGAFMV

SEQ ID NO: 4 - protein, 236 aa, Homo sapiens
  misc_feature: human mutant-LIGHT
MEESVVRPSVFVVDGQTDIPFTRLGRSHRRQSCSVARVGLGLLLLLMGAGLAVQGWFLLQLHWRLGEMVTRLPDGPAGSWQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV